(19) United States
(12) Patent Application Publication
(10) Pub. No.: US 2025/0243442 A1
(43) Pub. Date: Jul. 31, 2025

(54) SCAFFOLDING SUBSTRATE FOR PLURIPOTENT STEM CELLS, CELL CULTURE VESSEL, AND METHOD OF CULTURING PLURIPOTENT STEM CELLS

(71) Applicant: KABUSHIKI KAISHA TOYOTA CHUO KENKYUSHO, Nagakute-shi (JP)

(72) Inventors: Minoru HIRANO, Nagakute-shi (JP); Shougo Higashi, Nagakute-shi (JP)

(73) Assignee: KABUSHIKI KAISHA TOYOTA CHUO KENKYUSHO, Nagakute-shi (JP)

(21) Appl. No.: 19/035,190

(22) Filed: Jan. 23, 2025

(30) Foreign Application Priority Data
Jan. 31, 2024 (JP) 2024-013478

(51) Int. Cl. C12M 1/12; C12N 5/00

(52) U.S. Cl. CPC C12M 25/14; C12N 5/0068; C12N 2533/10; C12N 2535/00

(57) ABSTRACT

A scaffolding substrate for pluripotent stem cells including a fiber structure including textile fibers arranged three-dimensionally. The textile fibers include, at least on a surface thereof, a three-dimensional nanostructure in which metal-containing particles are arranged three-dimensionally. The metal-containing particles include at least one of a metal and a metal compound and include a particle diameter of not less than 1 nm and not greater than 60 nm.

### CROSS REFERENCE TO RELATED APPLICATIONS

The present application claims priority from Japanese patent application 2024-013478 filed on Jan. 31, 2024, the entirety of the content of which is hereby incorporated by reference into this application.

### TECHNICAL FIELD

The present disclosure relates to a scaffolding substrate for pluripotent stem cells, a cell culture vessel, and a method of culturing pluripotent stem cells.

### BACKGROUND

Pluripotent stem cells (for example, ES cells and iPS cells) in the undifferentiated state are used in the field of regenerative medicine and in a variety of academic fields. There has accordingly been a demand for a technique of culturing pluripotent stem cells of higher quality. A method of on-feeder culture using mouse fetus fibroblast cells as the feeder cell and a method of feeder-free culture using a plastic culture substrate coated with extracellular matrix (ECM) molecules (as described in, for example, Japanese Patent No. 7101377B and Japanese Patent No. 2022-77840A) have conventionally been known as the culture method of maintaining the pluripotent stem cells.

The on-feeder culture described above is a method of mixed culture with cells of a foreign species, whereas the feeder-free culture is a method of culture including a foreign species-derived protein that is derived from a different type of organism from an animal which the pluripotent stem cells are derived from. These known methods described above use the cells of foreign species or the foreign species-derived protein and accordingly have the risk of incorporation of a foreign virus or incorporation of the foreign species-derived protein. Especially in the regenerative medical settings, a culture method free from foreign constituents (xeno-free culture method) has been demanded. In other words, especially for the medical purposes, it is preferable that the constituents of the material used are chemically defined, and it is also preferable to suppress incorporation of undefined constituents derived from foreign species of organisms to a maximum extent.

An integrin molecule serving as an ECM receptor is provided on the surface of pluripotent stem cells. For example, the feeder-free culture described above enables the pluripotent stem cell to recognize the ECM molecule coating on a culture substrate and to be bonded to the substrate. This integrin-ECM bond gives a “survival” signal to the pluripotent stem cell, so that the pluripotent stem cell avoids anoikis, which is anchorage-dependent cell death. This integrin-ECM bond is a protein-protein interaction and is implemented when integrin recognizes an RGD sequence that is an amino acid sequence characteristic of ECM. Such integrin-ECM interaction is provided by specific three-dimensional conformations of both the proteins and by the electrostatic interaction between both the proteins. Accordingly, there has been no known method of providing a protein-free and peptide-free culture substrate for pluripotent stem cells, which is comprised of a material that has chemically defined constituents and that is artificially adjusted.

### SUMMARY

The present disclosure relates to a scaffolding substrate for pluripotent stem cells. The scaffolding substrate includes a fiber structure including textile fibers arranged three-dimensionally. The textile fibers include, at least on a surface thereof, a three-dimensional nanostructure in which metal-containing particles are arranged three-dimensionally. The metal-containing particles include at least one of a metal and a metal compound and include a particle diameter of not less than 1 nm and not greater than 60 nm.

The present disclose also relates to a cell culture vessel provided with the scaffolding substrate for pluripotent stem cells, a method of culturing pluripotent stem cells, and a method of manufacturing the scaffolding substrate for pluripotent stem cells.

### BRIEF DESCRIPTION OF DRAWINGS

FIG. 1 is an explanatory diagram schematically illustrating the configuration of a scaffolding substrate for pluripotent stem cells;

FIG. 2 is a flowchart showing one example of a method of manufacturing the scaffolding substrate for pluripotent stem cells;

FIG. 3 is a perspective view schematically illustrating the appearance of a cell culture vessel;

FIG. 4 is a schematic sectional view illustrating the state of culturing pluripotent stem cells by using the cell culture vessel;

FIG. 5 is an explanatory diagram schematically illustrating a method of culturing the pluripotent stem cells;

FIG. 6 is an explanatory diagram illustrating an estimated mechanism that allows for culturing the pluripotent stem cells;

FIG. 7 is an explanatory diagram illustrating surface images of respective samples observed by using a SEM;

FIG. 8 is an explanatory diagram showing fibers of a sample S1 observed by using a STEM;

FIG. 9 is an explanatory diagram showing the results of checking an element distribution on the surface of an NST by EDX;

FIG. 10 is an explanatory diagram showing the results of checking an element distribution on the surface of an NST by EDX;

FIG. 11 is an explanatory diagram showing the results of checking an element distribution on the surface of an NST by EDX;

FIG. 12 is an explanatory diagram showing the results of checking an element distribution on the surface of an NST by EDX;

FIG. 13 is an explanatory diagram showing the results of culturing iPS cells and checking whether the iPS cells were kept in the undifferentiated state;

FIG. 14 is an explanatory diagram showing the results of checking whether the iPS cells were kept in the undifferentiated state after reseeding on a “base material (I)”;

FIG. 15 is an explanatory diagram showing the results of checking whether the iPS cells were kept in the undifferentiated state after reseeding on the same base material as that for first passage; and

FIG. 16 is an explanatory diagram showing the results of examining the adhesion and growth and the results of verifying the undifferentiation in each passage number.

### DETAILED DESCRIPTION

The present disclosure may be implemented by the following aspects.
(1) According to one aspect of the present disclosure, there is provided a scaffolding substrate for pluripotent stem cells. This scaffolding substrate for pluripotent stem cells is formed as a fiber structure where fibers of textile are arranged three-dimensionally, wherein the textile has, on a surface thereof, a three-dimensional nanostructure in which metal-containing particles that are made of at least one of a metal and a metal compound and that have a particle diameter of not less than 1 nm and not greater than 60 nm, are arranged three-dimensionally.

In the scaffolding substrate for pluripotent stem cells of this aspect, the textile configuring the fiber structure has, on the surface thereof, the three-dimensional nanostructure in which the metal-containing particles having the particle diameter of not less than 1 nm and not greater than 60 nm are arranged three-dimensionally. This configuration enables the pluripotent stem cells to be cultured with suppressing incorporation of foreign constituents of foreign viruses and the like by using a protein-and peptide-free scaffolding substrate for cell culture, which is comprised of a material that has chemically defined constituents and that is artificially adjusted.
(2) In the scaffolding substrate for pluripotent stem cells of the above aspect, the three-dimensional nanostructure may have a primary structure configured by the metal-containing particles having the particle diameter of not less than 5 nm and not greater than 10 nm; and a secondary structure configured by a cluster that is an aggregate of the metal-containing particles and that has a diameter of not less than 30 nm and not greater than 100 nm. This configuration enhances the performance of allowing the pluripotent stem cells to be cultured while keeping the pluripotent stem cells in the undifferentiated state.
(3) In the scaffolding substrate for pluripotent stem cells of the above aspect, the fibers of the textile may have a diameter in cross section of not less than 100 nm and not greater than 700 nm. This configuration facilitates the pluripotent stem cells in ensuring the sufficient contact area with the textile in the scaffolding substrate for pluripotent stem cells and enables the pluripotent stem cells to recognize a microstructure at the nano level. This configuration enhances the adhesiveness of the pluripotent stem cells to the scaffolding substrate for pluripotent stem cells and enables the pluripotent stem cells to be grown favorably.
(4) In the scaffolding substrate for pluripotent stem cells of the above aspect, the fiber structure may be formed by two or more layers of the textile that are laid one upon another. This configuration increases the efficiency of supplies of oxygen, nutrient content and the like to the pluripotent stem cells that are cultured on the scaffolding substrate for pluripotent stem cells. This accordingly improves the growth of the pluripotent stem cells.
(5) In the scaffolding substrate for pluripotent stem cells of the above aspect, an interval between the fibers of the textile configuring the fiber structure may be not less than 0.5 μm and not greater than 10 μm. This configuration enables the individual pluripotent stem cells to be securely held on the scaffolding substrate for pluripotent stem cells.
(6) In the scaffolding substrate for pluripotent stem cells of the above aspect, the fibers of the textile may be formed in a tubular form or in a semi-tubular form. This configuration enhances the flexibility of the entire fiber structure and facilitates handling of the scaffolding substrate for pluripotent stem cells.
(7) In the scaffolding substrate for pluripotent stem cells of the above aspect, a metal element configuring the metal-containing particles may include at least one element among titanium (Ti), zirconium (Zr), aluminum (Al) and platinum (Pt). This configuration enhances the performance of allowing the pluripotent stem cells to be cultured while keeping the pluripotent stem cells in the undifferentiated state.
(8) In the scaffolding substrate for pluripotent stem cells of the above aspect, the metal element configuring the metal-containing particles may include at least one element out of titanium (Ti) and aluminum (Al). This configuration enhances the performance of allowing the pluripotent stem cells to be cultured while keeping the pluripotent stem cells in the undifferentiated state.
(9) In the scaffolding substrate for pluripotent stem cells of the above aspect, the metal element configuring the metal-containing particles may include titanium (Ti). This configuration enhances the performance of allowing the pluripotent stem cells to be cultured while keeping the pluripotent stem cells in the undifferentiated state, and more specifically, enhances the performance of allowing for passage culture with keeping the pluripotent stem cells in the undifferentiated state.

The present disclosure may be implemented by a variety of aspects other than those described above: for example, a cell culture vessel provided with the scaffolding substrate for pluripotent stem cells, a method of culturing pluripotent stem cells, and a method of manufacturing the scaffolding substrate for pluripotent stem cells.

### A. Configuration of Scaffolding Substrate for Pluripotent Stem Cells

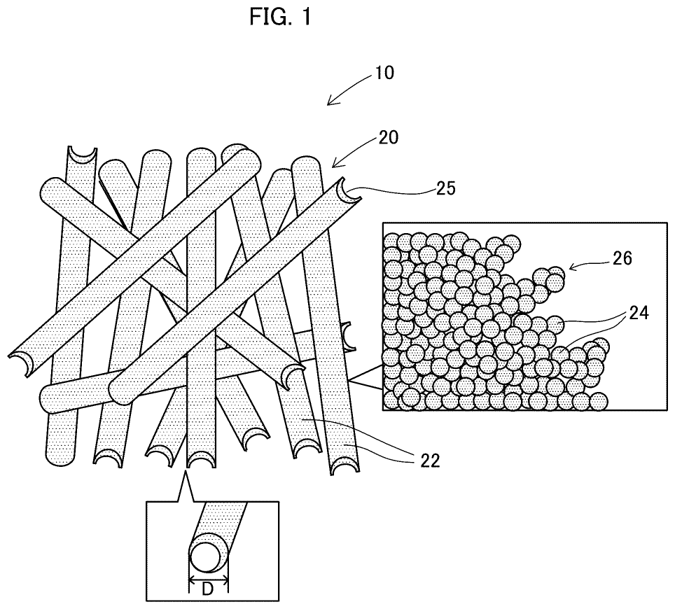

FIG. 1 is an explanatory diagram schematically illustrating the configuration of a scaffolding substrate for pluripotent stem cells 10 according to one embodiment of the present disclosure. The scaffolding substrate for pluripotent stem cells 10 of the embodiment denotes a scaffolding substrate used for culturing pluripotent stem cells that are animal cells, such as ES cells and iPS cells, which are in an undifferentiated state and which have multipotency or pluripotency to be differentiated into a variety of cells.

The scaffolding substrate for pluripotent stem cells 10 of the embodiment is formed as a fiber structure 20 where fibers of textile 22 are arranged three-dimensionally. The textile 22 has, on a surface thereof, a three-dimensional nanostructure in which metal-containing particles 24 that are made of at least one of a metal and a metal compound and that have a particle diameter of not less than 1 nm and not greater than 60 nm, are arranged three-dimensionally. The particle diameter of the metal-containing particle 24 may be calculated as a relative value of a pixel distance from one end to the other end of an observed object (metal-containing particle 24) to the scale bar size in an image taken by using a scanning electron microscope (SEM) or a transmission electron microscope (TEM). As long as the textile 22 has the three-dimensional nanostructure described above on the surface thereof, for example, the entirety of the textile 22 may be configured by an aggregate of the metal-containing particles 24. In another example, the three-dimensional nanostructure described above may be formed on a substrate (base material) made of a different material from that of the metal-containing particles 24. The fiber structure 20 is preferably formed in a cloth form such as non-woven textile where the fibers of the textile 22 are linked with one another three-dimensionally. In the description below, the fiber structure 20 consisting of the textile 22 having, on the surface thereof the three-dimensional nanostructure, which is configured by the metal-containing particles 24 (hereinafter also referred to as “nanoparticles”), is also called nanostructured textile (NST).

The three-dimensional nanostructure on the surface of the textile 22 may have a primary structure configured by the metal-containing particles 24 having the particle diameter of not less than 5 nm and not greater than 10 nm; and a secondary structure configured by a cluster that is an aggregate of such metal-containing particles 24 and that has a diameter of not less than 30 nm and not greater than 100 nm. FIG. 1 illustrates formation of the three-dimensional nanostructure having the primary structure and the secondary structure described above, in closeup of the surface of the textile 22.

The metal-containing particles 24 configuring the scaffolding substrate for pluripotent stem cells 10 may be composed of a single metal element, may be composed of an alloy, may be composed of a metal compound, or may be composed of a mixture of the foregoing. The metal-containing particles 24 may be crystalline or may be non-crystalline or amorphous. The metal compound configuring the metal-containing particles 24 may be at least one type among, for example, metal oxides, metal sulfides, metal nitrides, metal carbides, metal phosphides, and metal iodides. The metal compound is preferably a metal oxide.

The metal element configuring the metal-containing particles 24 may be, for example, a titanium group element (group IV (4) element), such as titanium (Ti) or zirconium (Zr) or a transition metal element, such as vanadium (V), chromium (Cr), manganese (Mn), iron (Fe), cobalt (Co), nickel (Ni), copper (Cu), zinc (Zn), yttrium (Y), niobium (Nb), or molybdenum (Mo). The metal element configuring the metal-containing particles 24 may also be a platinum group element, such as platinum (Pt), ruthenium (Ru), rhodium (Rh), palladium (Pd), osmium (Os), or iridium (Ir) or a noble metal element, such as gold (Au) or silver (Ag). The metal element configuring the metal-containing particles 24 may otherwise be a typical metal element, such as aluminum (Al), magnesium (Mg), silicon (Si), gallium (Ga), germanium (Ge), arsenic (As), selenium (Se), indium (In), tin (Sn), antimony (Sb) or tellurium (Te). Among them, the metal element configuring the metal-containing particles 24 preferably includes at least one element among titanium (Ti), zirconium (Zr), aluminum (Al) and platinum (Pt) and more preferably includes at least one element out of titanium (Ti) and aluminum (Al). In terms of favorable passage culture of pluripotent stem cells by using the scaffolding substrate for pluripotent stem cells 10, the metal element configuring the metal-containing particles 24 especially preferably includes titanium (Ti).

For example, the textile 22 may be produced by using, as a substrate (base material), a fibrous body made of a material different from the constituent material of the metal-containing particles 24 and providing a layer of the metal-containing particles 24 having the three-dimensional nanostructure on the surface of the fibrous body as the base material. The layer of the metal-containing particles 24 may be formed on the base material by, for example, vapor deposition such as physical vapor deposition (PVD) or chemical vapor deposition (CVD) or atomic layer deposition (ALD) and is preferably formed by physical vapor deposition. The object (base material) for formation of the layer described above may be, for example, a cloth-like member such as a non-woven textile formed from a fibrous body made of a different material from the constituent material of the metal-containing particles. More specifically, a fibrous body made of a resin may be used as the base material. Using a resin member as the base material enables the nucleation and the grain growth of the nanoparticles to proceed relatively easily on the surface of the base material, in the case of formation of the layer of the metal-containing particles 24 by physical vapor deposition or the like. For example, the presence of oxygen other than the metal element during vapor deposition allows for formation of a metal layer or a metal oxide layer according to the type of the metal element used. In the process of producing the textile 22 in this manner, the wraparound of the vapor deposition material occurs during the vapor deposition. This causes a tubular layer or a semi-tubular layer to be formed as the layer of the metal-containing particles 24 having the three-dimensional nanostructure on the surface of the fibrous body as the base material. Formation of layers from both surfaces of the fibrous body as the base material ensures formation of a tubular layer with high accuracy.

For example, physical vapor deposition of the constituent material of the metal-containing particles 24 on the surface of the fibrous body as the base material causes nucleation and grain growth of a large number of nanoparticles on the surface of the base material. Continuation of the physical vapor deposition repeats the nucleation and the grain growth of the nanoparticles on the surface of the fibrous body. As a result, a cluster 26 is formed as an aggregate of the metal-containing particles 24 on the surface of the fibrous body, and the structure having both the primary structure and the secondary structure described above is formed as the three-dimensional nanostructure. The cluster 26 is formed, for example, in a salient form having a conical outer shape such as a polygonal pyramidal shape or a cone shape. The diameter of this cluster 26 may be, for example, not less than 30 nm and not greater than 100 nm. The “diameter of the cluster 26” denotes a maximum diameter of the salient form (for example, a diameter of a bottom face in the case of a cone). The diameter of the cluster 26 may be calculated by using an image of SEM or TEM by a method similar to the method of calculating the particle diameter of the metal-containing particles 24 described above. The diameters and the number of the salient structures may be controlled, depending on the conditions of vapor deposition.

After formation of the layer having the three-dimensional nanostructure on the surface of the fibrous body as the base material, the base material may be removed completely, may be left partly, or may be left entirely. When the constituent material of the fibrous body as the base material is a material that is not dissolved in a culture medium for cell culture or is a material that has no cytotoxic activity, at least part of the fibrous body described above may be left in the scaffolding substrate for pluripotent stem cells 10. FIG. 1 illustrates, as an example, the fiber structure 20 obtained by forming a semi-tubular layer as the layer having the three-dimensional nanostructure on the surface of the fibrous body as the base material and subsequently removing the base material. A base material space 25 is provided after removal of the base material in the fibers of the textile 22 formed in the semi-tubular shape, like the textile 22 of the fiber structure 20 shown in FIG. 1. Using the textile 22 made of the metal or the alloy with removal of the base material as described above enhances the flexibility of the entire fiber structure 20 and facilitates handling of the fiber structure 20.

The fibers of the textile 22 configuring the scaffolding substrate for pluripotent stem cells 10 of the embodiment has a diameter (average diameter) in cross section of preferably not less than 100 nm, of more preferably not less than 150 nm and of furthermore preferably not less than 200 nm. The fibers of the textile 22 have the diameter (average diameter) in cross section of preferably not greater than 700 nm, of more preferably not greater than 650 nm, and of furthermore preferably not greater than 600 nm. The diameter of the fibers of the textile 22 may be calculated by using an image of SEM or TEM by a method similar to the method of calculating the particle diameter of the metal-containing particles 24 described above. The diameter of the fibers of the textile 22 may be regulated by the diameter of the fibrous body as the base material for formation of the layer having the three-dimensional nanostructure or by conditions of layer formation using the fibrous body as the base material. In the case of formation of the fibers of the textile 22 in the semi-tubular shape, which configures the fiber structure 20 (in the case of formation of the fibers of the textile 22 having a cross section in a partly missing shape, such as a crescent shape), the diameter of the fibers of the textile 22 denotes a diameter of a virtual circle formed by virtually filling the missing part of the cross section (as shown by a diameter D in FIG. 1). A procedure of determining the average diameter of the fibers of the textile 22 may, for example, take an image of the fiber structure 20 by using a scanning electron microscope (SEM), observe a predetermined sufficient number of fields of view (for example, five fields of view), determine the diameter of the fibers of the textile 22 selected in each of the fields of view, and calculate an average value of the diameters measured in the respective fields of views, as the average diameter of the fibers of the textile 22.

The fiber structure 20 serving as the scaffolding substrate for pluripotent stem cells 10 needs to have, for example, one or multiple layers of the textile 22 and may be formed by multiple layers of the textile 22 laid one upon another in a thickness direction, i.e., may be formed by two or more layers of the textile 22 that are laid one upon another. The number of the layers of the textile 22 configuring the fiber structure 20 may be, for example, not greater than 300 layers, may be not greater than 200 layers or may be not greater than 100 layers. In this fiber structure 20, the interval between the fibers of the textile 22 configuring the fiber structure 20 may be not less than 0.5 μm and not greater than 10 μm. When the shape of the fiber structure 20 is recognized as a stacked shape of stacking mesh structures formed by crossing three fibers of the textile 22 to form a triangle a shape as a minimum unit, the “interval between fibers of textile” herein is defined as a length of a long side (longest side) of the triangle.

### B. Method of Manufacturing Scaffolding Substrate for Pluripotent Stem Cells

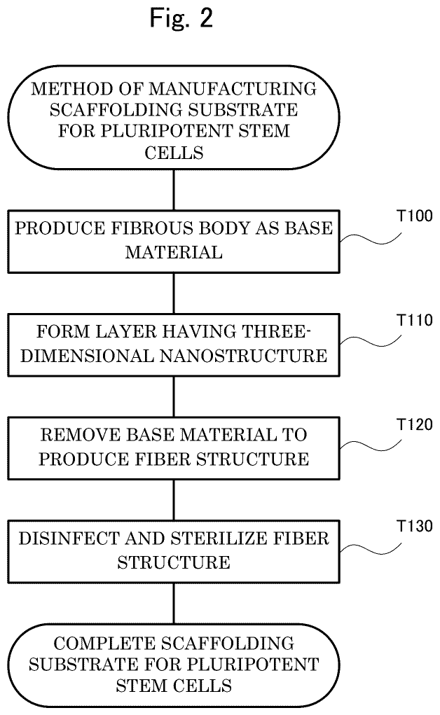

FIG. 2 is a flowchart showing one example of a method of manufacturing the scaffolding substrate for pluripotent stem cells 10. The following describes the method of manufacturing the scaffolding substrate for pluripotent stem cells 10 to form the layer having the three-dimensional nanostructure on the surface of the fibrous body as the base material by sputtering that is a physical vapor deposition.

The method of manufacturing the scaffolding substrate for pluripotent stem cells 10 first produces a fibrous body as a base material (process T100). The fibrous body may be produced by, for example, electrospinning. The electrospinning that is one of techniques of producing nanofibers is a known technique of spinning any of a variety of materials including resins (polymers) into nanofibers, and enables a non-woven textile to be produced directly. More specifically, a procedure places a polymer solution or a molten polymer into a syringe, ejects the polymer solution under application of a high voltage to generate a jet flow of the charged polymer, and collects this jet flow to form extra-fine polymer fibers. The power voltage may be regulated appropriately according to the type of the polymer solution to be charged and the desired thickness of the polymer fibers. The fiber diameter of the produced fibrous body may be adjusted, for example, by regulating the polymer concentration of the solution used for electrospinning, the electric field, and the supply speed of the solution.

A variety of polymers may be used as the base material. Examples of the polymers usable include polyether sulfone (PES), polyvinylidene fluoride (PVDF), polyvinylpyrrolidone (PVP), polyethylene (PE), polypropylene (PP), polyester, polyvinyl alcohol (PVA), polyacrylonitrile (PAN), polyethylene oxide (PEO), polyacrylate (ACM), and polypropylene oxide (PPO).

After producing the fibrous body at the process T100, the method forms a layer having a three-dimensional nanostructure on the surface of the produced fibrous body (process T110). A variety of techniques may be employed as the technique for forming the layer having the three-dimensional nanostructure as described above. The physical vapor deposition technique, such as the sputtering technique or the pulse layer deposition technique is preferable, and the sputtering technique is especially preferable. In this process, the layer described above may be formed in an inert gas under reduced pressure or may be formed in a gas phase containing oxygen. A variety of metal elements described above as the constituent material of the metal-containing particles 24 may be used as the material for formation of the layer having the three-dimensional nanostructure. The conditions for formation of the layer having the three-dimensional nanostructure are not specifically limited but may be appropriately adjusted, depending on the purpose. In the case of employing physical vapor deposition, in general, the longer vapor deposition time causes the larger thickness of the layer formed. The physical vapor deposition technique can control the amount of vapor deposition at an atomic level, so that a desired size of the cluster 26 can be formed by optimizing the conditions of vapor deposition. A semi-tubular layer or a tubular layer may be formed as the layer of the metal-containing particles 24 having the three-dimensional nanostructure by vapor deposition from a single face of or from both faces of the base material.

After forming the layer having the three-dimensional nanostructure at the process T110, the method removes the base material to produce the fiber structure 20 (process T120). The technique for removing the base material is not specifically limited but may be, for example, firing or calcination. The firing or calcination may be performed at such a temperature that decomposes and removes the base material (resin material). Depending on the material of the metal-containing particles 24, the shape of the cluster 26 may further be adjusted in this firing process. In the case where a solvent-soluble polymer is used as the base material, the base material may be removed by dissolving the base material in a solvent. Examples of the solvent usable to dissolve a variety of polymers include dimethylformamide (DMF), N-methyl-2-pyrollidone (NMP), a sodium borohydride (NaBH4) solution using a 1 to 1 mixed liquid of water and ethanol as a solvent, chloroform, acetone, alcohols such as methanol and ethanol, water, 2-methyltetrahydrofuran, dioxane, dimethylsulfoxide, sulfolane, and nitromethane. Only part of the base material may be removed at the process T120 as described above. The process T120 may not be performed at all, in order to produce the fiber structure 20 while leaving the base material.

After the process T120, the method disinfects or sterilizes the obtained fiber structure 20 (process T130), so as to complete the scaffolding substrate for pluripotent stem cells 10.

The method of manufacturing the scaffolding substrate for pluripotent stem cells 10 provided with the metal-containing particles 24 by a gas phase technique such as physical vapor deposition does not need a process of, for example, collecting, cleaning and drying nanoparticles and also does not need equipment for safely handling the nanoparticles as in the case of solution phase synthesis of nanoparticles. This configuration accordingly enables the scaffolding substrate for pluripotent stem cells that is the structure provided with the nanoparticles to be manufactured more easily. This manufacturing method also enables the large-area scaffolding substrate for pluripotent stem cells 10 to be readily manufactured.

### C. Cell Culture Vessel

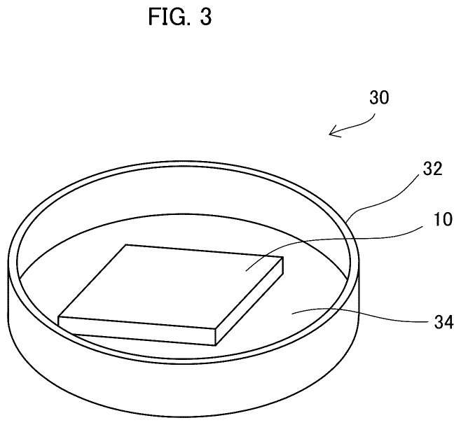

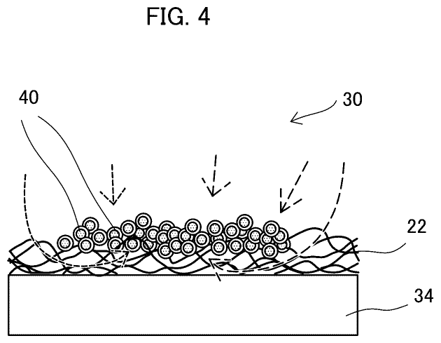

FIG. 3 is a perspective view schematically illustrating the appearance of a cell culture vessel 30 according to the embodiment. FIG. 4 is a schematic sectional view illustrating the state of culturing pluripotent stem cells 40 by using the cell culture vessel 30. The cell culture vessel 30 is provided with the scaffolding substrate for pluripotent stem cells 10 according to the embodiment in at least part of a culturable area in a vessel main body 32 that configures the cell culture vessel 30. This cell culture vessel 30 is preferably usable for culturing pluripotent stem cells.

The shape of the vessel main body 32 configuring the cell culture vessel 30 is not specifically limited but may be, for example, a bottomed tubular shape having a bottom face 34 that is a substrate portion forming a culturable area where the scaffolding substrate for pluripotent stem cells 10 is placed. The vessel main body 32 shown in FIG. 3 has a bottomed cylindrical shape with a circular shape in top view but may have a different shape. The vessel main body 32 may be formed in a plate-like shape having a plurality of wells in a bottomed tubular shape and may be configured to place the scaffolding substrate for pluripotent stem cells 10 in a bottom face of each of the wells. The number of the wells, the size of each well and the shape in top view of each well may be set appropriately according to, for example, the purpose of the culture.

The material of the vessel main body 32 is not specifically limited but may be, for example, a resin material, an inorganic material, or a metal material. Examples of the resin material usable include polystyrene, polyethylene, polycarbonate, polypropylene, acrylic resin, and silicone resin. Examples of the inorganic material usable include glass and titanium oxide. Examples of the metal material usable include stainless steel, nickel and titanium.

### D. Method of Culturing Pluripotent Stem Cells

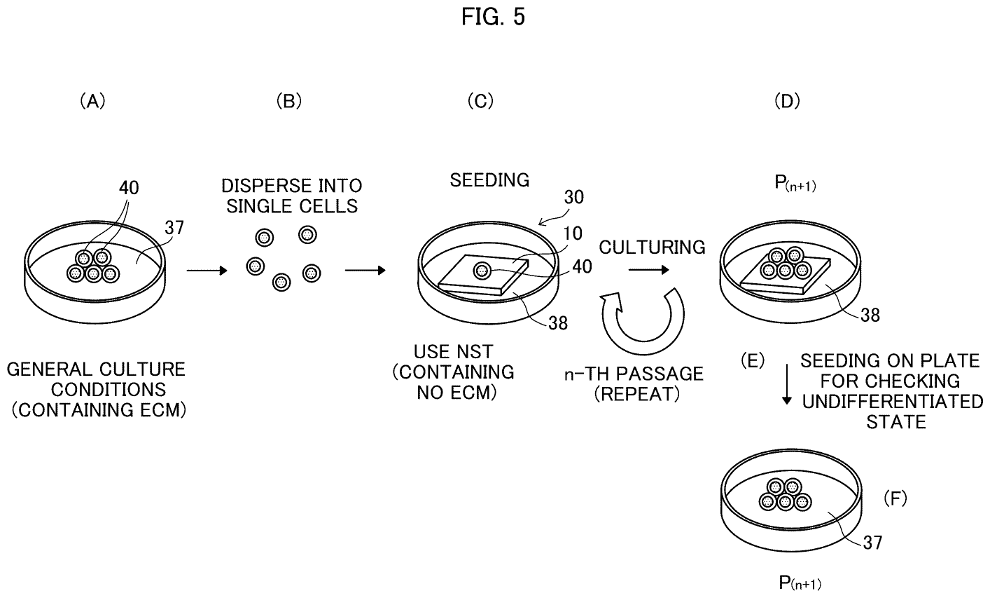

FIG. 5 is an explanatory diagram schematically illustrating one example of a method of culturing the pluripotent stem cells 40 by using the cell culture vessel 30 provided with the scaffolding substrate for pluripotent stem cells 10 according to the embodiment. The method of culturing the pluripotent stem cells 40 by using the cell culture vessel 30 shown in FIG. 5 first provides the pluripotent stem cells 40 by culture not using the scaffolding substrate for pluripotent stem cells 10 but using an ECM-containing general culture medium for undifferentiated cells 37 (process A). The method subsequently disperses the provided pluripotent stem cells 40 in the state of single cells or in the state of clumps of cells (process B), and seeds the individual pluripotent stem cells 40 on the scaffolding substrate for pluripotent stem cells 10 in the cell culture vessel 30 (process C). The method then uses an ECM-free culture medium 38 that substantially contains no ECM to continue culturing until a confluent state (process D). For example, the operation of dispersing the pluripotent stem cells 40 in the process B may perform an enzyme treatment or may use a calcium chelating agent such as EDTA, so as to peel the pluripotent stem cells 40 from the scaffolding substrate for pluripotent stem cells 10.

The pluripotent stem cells 40 through the culture in the process D described above are cultured by using the ECM-free culture medium 38 and can thus be used for a variety of experiments and the like, as cells cultured in such an environment that has chemically defined constituents and that is artificially adjusted. In the case of continuing the culture using the ECM-free culture medium 39 for a longer time period, an operation of redispersing the pluripotent stem cells 40 sufficiently grown in the process D and seeding (reseeding) the individual pluripotent stem cells 40 on a new cell culture vessel 30 may be repeated (process E). In order to check whether the cells cultured in the process D are kept in the undifferentiated state, for example, one available method may seed the cells cultured in the process D on a culture plate including the ECM-containing general culture medium for undifferentiated cells 37 (i.e., a plate for checking the undifferentiated state) (process F) and may make a check, for example, by a staining technique using an undifferentiation marker.

In the scaffolding substrate for pluripotent stem cells 10 of the embodiment configured as described above, the textile 22 configuring the fiber structure 20 have, on the surface thereof, the three-dimensional nanostructure where the metal-containing particles 24 having the particle diameter of not less than 1 nm and not greater than 60 nm are arranged three-dimensionally. This configuration enables the pluripotent stem cells 40 to be cultured while suppressing incorporation of foreign constituents by using a protein-and peptide-free scaffolding substrate for cell culture, which is comprised of a material that has chemically defined constituents and that is artificially adjusted. More specifically, this configuration enables the pluripotent stem cells 40 to be grown by using a scaffolding substrate for cell culture that contains no intrinsic ECM proteins such as laminin, fibronectin, and vitronectin and partial sequencing peptides thereof and that is comprised of a material which has chemically defined constituents and which is artificially adjusted.

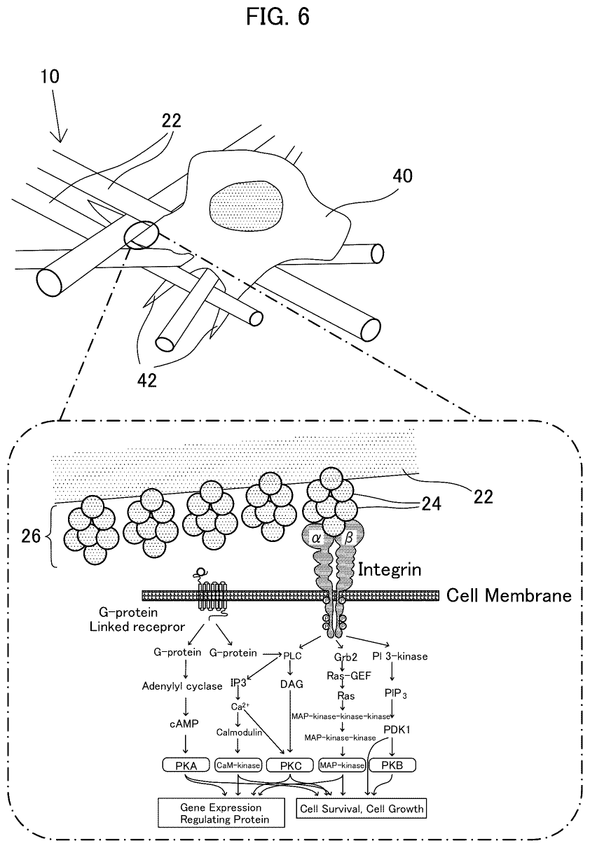

FIG. 6 is an explanatory diagram illustrating an estimated mechanism that allows for culturing the pluripotent stem cells 40 by using the scaffolding substrate for pluripotent stem cells 10. FIG. 6 illustrates the state in which the pluripotent stem cells 40 are cultured on the scaffolding substrate for pluripotent stem cells 10, while extending filopodia 42 between the fibers of the textile 22. FIG. 6 also shows the closeup of a contact region between the pluripotent stem cell 40 and the textile 22 to indicate an estimated mechanism relating to the interaction between the pluripotent stem cell 40 and the textile 22.

A pluripotent stem cell, such as an embryonic stem cell (ES cell) or an induced pluripotent stem cell (iPS cell), originally recognizes an electrostatic distribution and a three-dimensional nanostructure (three-dimensional conformation including an RGD sequence and having the size of about 5 to 10 nm) of a specific amino acid sequence in an ECM molecule via an integrin molecule on the surface of the pluripotent stem cell, and bonds to the three-dimensional nanostructure to escape anchorage-dependent cell death (anoikis). In the scaffolding substrate for pluripotent stem cells 10 of the embodiment, the three-dimensional nanostructure on the surface of the textile 22 comprised of the artificially adjusted material simulates the cell scaffolding microenvironment described above. It is thought that this causes the pluripotent stem cells 40 to falsely recognize the surface structure of the textile 22 as intrinsic ECM molecules, to escape the anoikis, and to bond and grow as normal undifferentiated pluripotent stem cells.

Especially the configuration of culturing the pluripotent stem cells 40 on the scaffolding substrate for pluripotent stem cells 10 of the embodiment, which is formed to have a network structure by the plurality of fibers of the textile 22 laid one upon another in the thickness direction, obtains the effect of enhancing the growth of the pluripotent stem cells 40. More specifically, using the scaffolding substrate for pluripotent stem cells 10 enables the pluripotent stem cells 40 to receive the supplies of oxygen as well as of the nutrient content, the proteinaceous growth factors and the like in the culture medium not only from an upper side but from a lower side of the pluripotent stem cells 40 via a space in the network structure of the base material as shown in FIG. 4 and thereby enhances the growth. In FIG. 4, the state of supplies of oxygen, the nutrient content and the like to the pluripotent stem cells 40 is shown by broken line arrows. In terms of ensuring the efficiency of the supplies of oxygen, the nutrient content and the like from the lower side of the pluripotent stem cells 40, neither the number of layers of the textile 22 that are laid one upon another in the scaffolding substrate for pluripotent stem cells 10 nor the thickness of the scaffolding substrate for pluripotent stem cells 10 is specifically limited.

In this scaffolding substrate for pluripotent stem cells 10, the interval between the fibers of the textile 22 configuring the fiber structure 20 is preferably not less than 0.5 μm and not greater than 10 μm. This configuration enables each of the pluripotent stem cells 40 to be securely held on the surface of the fiber structure 20. Sufficiently limiting the diameter in the cross section of the fibers of the textile 22 to, for example, not less than 100 nm and not greater than 700 nm facilitates the pluripotent stem cell 40 to extend the filopodia 42 and to ensure the sufficient contact area with the scaffolding substrate for pluripotent stem cells 10. This configuration enhances the adhesiveness of the pluripotent stem cells 40 to the scaffolding substrate for pluripotent stem cells 10 and enables the pluripotent stem cells 40 to be grown favorably.

### EXAMPLES

### Adjustment of Scaffolding Substrate for Pluripotent Stem Cells (NST)

### (Producing Fibrous Body as Base Material)

A non-woven textile (PVP non-woven textile) made of polyvinyl pyrrolidone (PVP) was produced as a fibrous body that was a base material for producing a nanostructured textile (NST). The PVP non-woven textile was produced by electrospinning on a conductive metal substrate made of, for example, titanium (Ti). More specifically, a methanol solution containing 8 wt % of PVP was set in a syringe, and electrospinning was performed under the following conditions: an applied voltage of 1 kV/cm, a solution sending rate of 1 mL/h, and a total solution sending volume of 0.4 mL. The size of the textile 22 obtained and the size of the interval between the fibers of the textile 22 are controllable by changing the type of fibers and the conditions of electrospinning (the concentration of the solution, the applied voltage, the distance from a solution sending port to the substrate, and the solution sending volume).

### (Producing NST Having Three-Dimensional Nanostructure)

The NST was produced by forming a three-dimensional nanostructure on the PVP non-woven textile that was the base material. Four NSTs were produced: a “sample S1” including titanium (Ti), a “sample S2” including zirconium (Zr), a “sample S3” including aluminum (Al), and a “sample S4” including platinum (Pt), as the metal included in the metal-containing particles 24 configuring the three-dimensional nanostructure. The “sample S1”, the “sample S2, the “sample S3” and the “sample S4” are also called “NST-Ti”, “NST-Zr”, “NST-Al”, and “NST-Pt”, respectively. The three-dimensional nanostructure of each of the samples was formed by sputtering as a physical vapor deposition technique.

The three-dimensional nanostructure of the sample S1 was formed by a sputtering procedure using the PVP non-woven textile as the base material and specifying Ti as a target element under an atmosphere including 0.56% of oxygen and 99.44% of argon. More specifically, after evacuation of a metal substrate with a PVP non-woven textile coated thereon, sputtering was performed by introducing argon at a flow rate of 30 sccm and oxygen at a flow rate of 0.17 sccm and regulating the internal pressure of a chamber to 10.0 Pa. The three-dimensional nanostructures of the samples S2 to S4 were similarly formed by specifying each corresponding metal element as the target element. The three-dimensional nanostructure of another type of metal can be formed by changing the target element and appropriately adjusting the gaseous species.

After the sputtering, the fibrous body with the three-dimensional nanostructure formed on the surface thereof was punched out to a size of 12 mm in diameter and was placed in water provided in advance in a container. A fiber structure with removal of PVP as the base material was obtained in water by cleaning the fibrous body with the three-dimensional nanostructure formed thereon. The obtained fiber structure was scooped with a Ti mesh, and a surface of the fiber structure, which was not in contact with the mesh, was applied on a glass substrate, so that the fiber structure was transferred onto the glass substrate. The glass substrate with the transferred fiber structure was then dried in an incubator at 70° C. for 5 minutes and was subjected to heating treatment in the air in a heating and firing furnace at 500° C. for 30 minutes. This completed the NST as each of the samples with removal of PVP. The primary structure and the secondary structure in the three-dimensional nanostructure of the NST are controllable by changing the metal species (the type of target) that is to deposit on the PVP non-woven textile, the conditions of sputter deposition (the composition of the atmosphere gas, the pressure, and the sputter deposition rate), the heating treatment conditions, and the like.

### (Confirming Microstructure of NST)

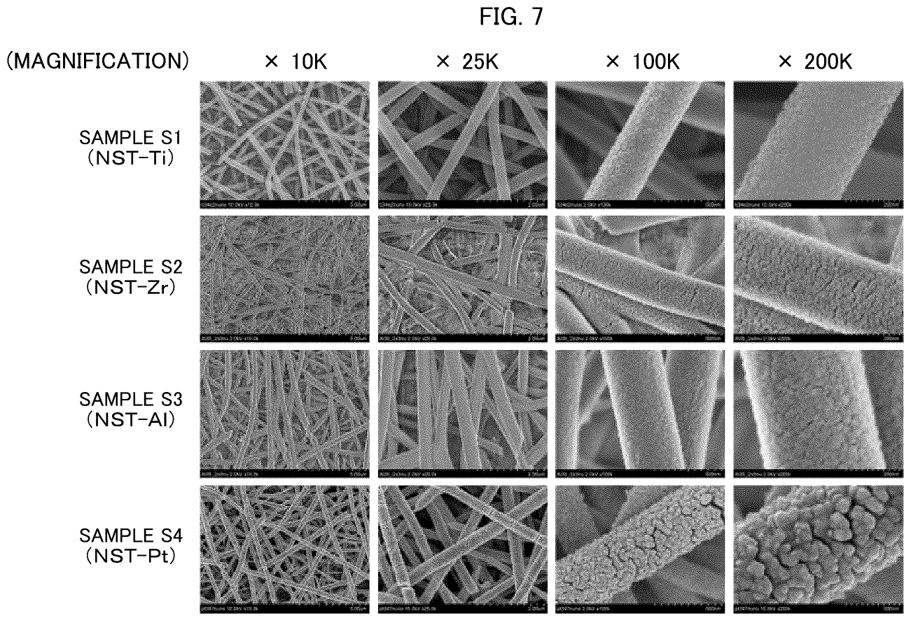

FIG. 7 is an explanatory diagram illustrating surface images of the respective produced samples observed by using a SEM (scanning electron microscope). As shown in FIG. 7, it was confirmed that each of the samples (NST) produced by electrospinning was a fiber structure that was dense in a vertical direction (in a thickness direction), irrespective of the target element. The constituent fibers were laid one upon another to form a meshed structure where three fibers as a minimum unit crossed one another to form a triangle (having the length of a longitudinal side or the interval between fibers in a range of 3 to 5 μm) and where these triangles were laid one upon another in a sterically complicated manner in the vertical direction. The diameter of each of the fibers in each of the samples was about 300 to 500 nm. The roughness of the particles formed on the surface of the fibers by sputtering after electrospinning depends on the metal species. In the samples S1 to S3, the particles of about 10 to 20 nm were formed. In the sample S4, the particles of about 20 to 500 nm were formed. An aspect ratio of each of the particles in a depth direction was almost 1 to 1. In the sample S4 (NST-Pt), however, the observation showed the state that a plurality of particles formed a cluster to be fixed in a fibrous manner and had crevasse-like deep valleys (about 60 nm).

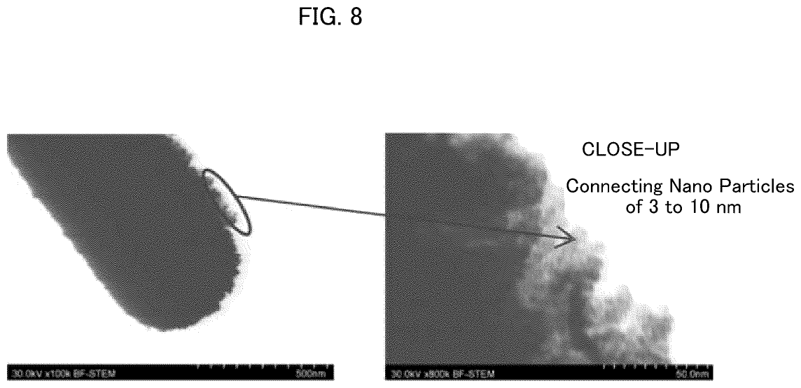

FIG. 8 is an explanatory diagram showing the fibers of the sample S1 (NST-Ti) observed at a larger magnification by using a scanning transmission electron microscope (STEM). The right side of FIG. 8 shows a closeup image of part of the surface of the fibers. The observation showed the state that the particles observed in the SEM image of FIG. 7 were configured as the cluster 26 that was an aggregate of primary particles (the metal-containing particles 24) having the particle diameter of about 3 to 10 nm.

### (Checking Element Distribution on Surface of NST by EDX)

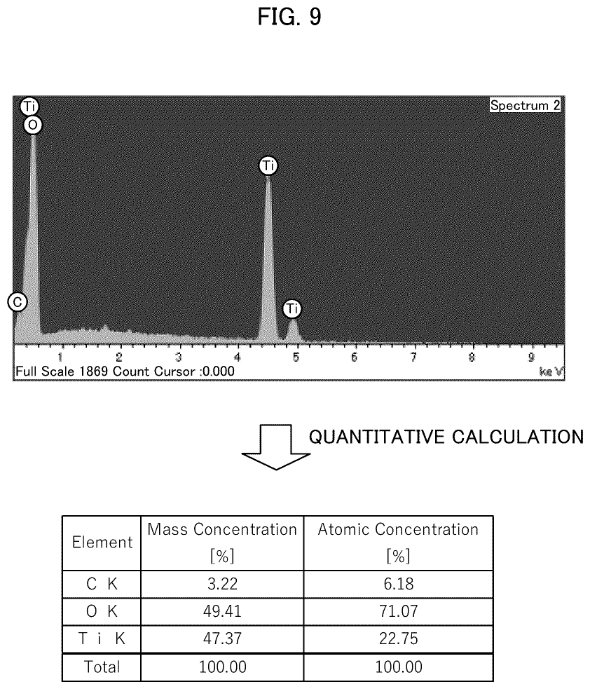

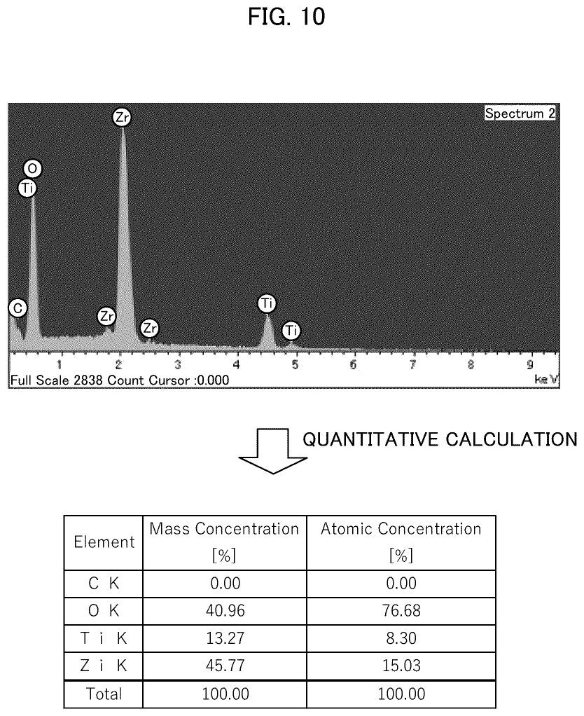

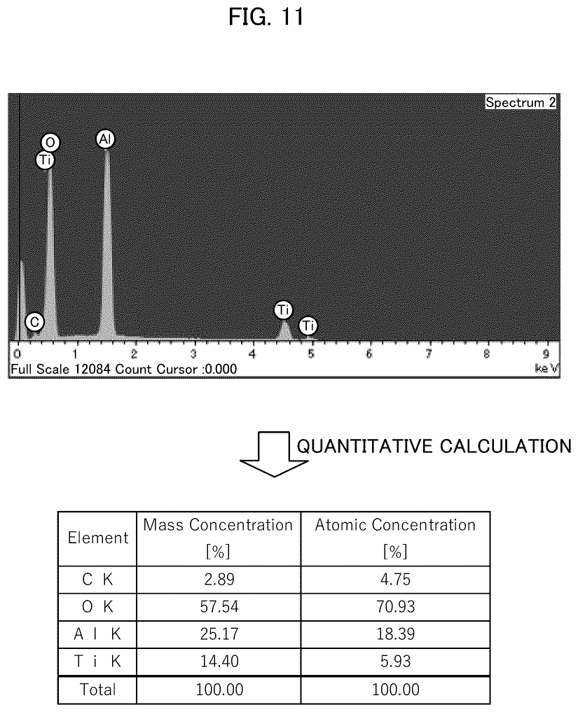

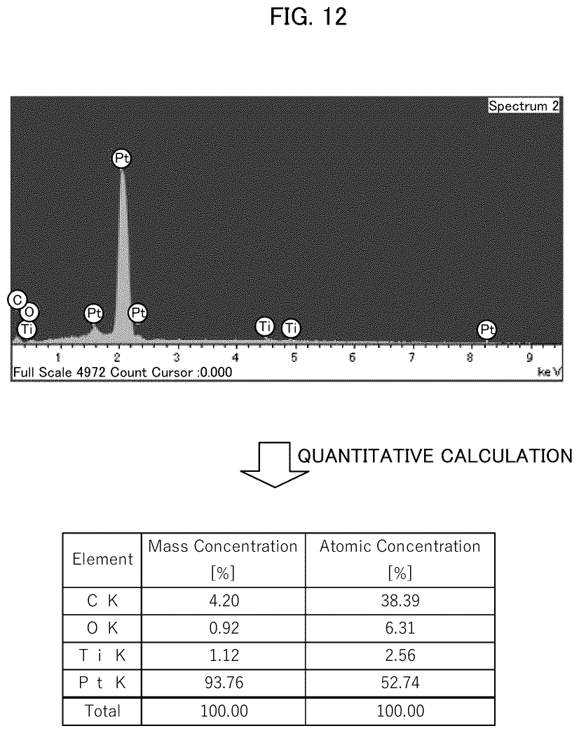

FIGS. 9 to 12 are explanatory diagrams showing the results of checking the element distribution on the surface of the NST in each of the samples by EDX. FIG. 9 shows the results of the sample 1; FIG. 10 shows the results of the sample 2; FIG. 11 shows the results of the sample 3; and FIG. 12 shows the results of the sample 4. In each of these diagrams, the upper side shows a qualitative analysis chart of EDX, and the lower side shows the results of quantitative calculation. In the results of quantitative calculation, the element column shows the element and the type of characteristic X ray.

As shown in FIGS. 9 to 12, when the surface of arbitrary fibers in each of the samples was analyzed by spot EDX analysis (energy dispersive X-ray fluorescence analysis), a metal element corresponding to a target element species of sputtering was detected in each of the samples. In each of the NSTs of the samples S1 to S3, a large amount of oxygen was detected. It is accordingly estimated that the metal element is present in the state of an oxide in these samples. In the sample S4 using platinum (Pt) as the target element species, on the other hand, a significantly small amount of oxygen was detected. It is accordingly estimated that platinum is sputtered as particles in the metallic state.

### Seeding of iPS Cells on NST and Evaluation

### (Pretreating and Sterilizing NST)

The procedure placed each of the produced samples (NSTs) in a well of a 24 multi-well plate and treated the sample with 70% ethanol for 15 minutes, in order to sterilize the bacteria present on the surface of the sample (NST). The procedure subsequently removed ethanol, rinsed the surface of the sample with sterilized water to completely remove the washing solution, and then air-dried the sample.

### (Seeding iPS Cells and Confirming iPS Cells Kept in Undifferentiated State)

The procedure provided iPS cells having passage ability (cells on the seventh day after seeding, cultured by using StemFit (manufactured by Ajinomoto Co., Ltd.) as a culture medium for cell culture and iMatrix-511 (manufactured by Nippi, Incorporated) as an ECM) (process A of FIG. 5), washed the surface of the provided iPS cells with PBS once and dissociated the cells into the state of single cells by using 0.5×TrypLE Express (Thermo Fisher Scientific Inc.) (process B of FIG. 5). The procedure subsequently suspended the dissociated cells at a density of 0.15×10⁴ cells/cm² in a 10 μM Y-27632-containing StemFit, seeded the suspended cells onto a variety of base materials including the samples S1 to S4 described above (process C of FIG. 5), and then cultured the cells in an ECM-free environment (process D of FIG. 5).

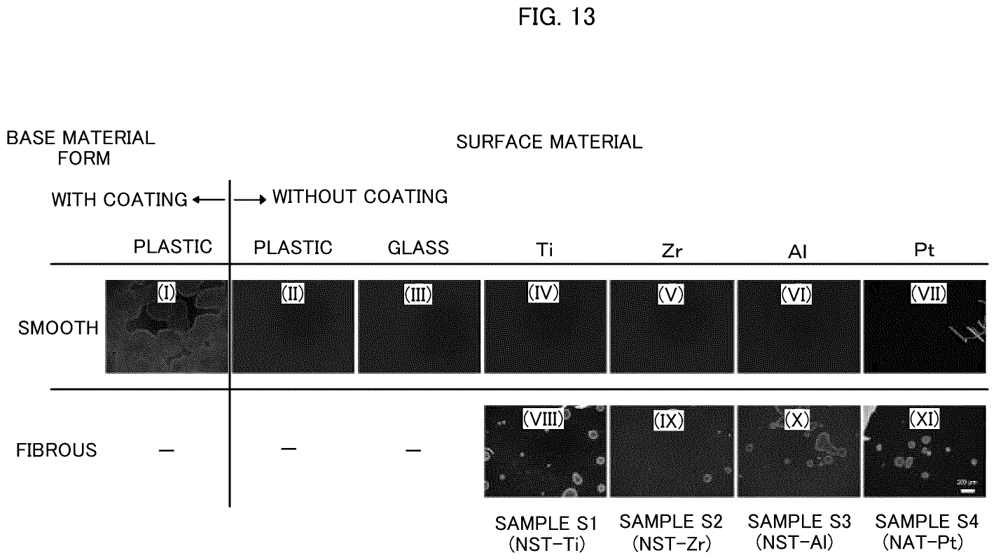

FIG. 13 is an explanatory diagram showing the results of culturing the iPS cells on a variety of base materials including the samples S1 to S4 and checking whether the iPS cells were kept in the undifferentiated state. A general plate for tissue culture was used as a plastic dish. A “base material (I)” in FIG. 13 was a control base material for seeding the iPS cells, which was obtained by coating the above plastic dish with iMatri-511 as the ECM. The base material had no ECM coating in “base materials (II) to (XI)” in FIG. 13. In FIG. 13, a “base material (II)” was a plastic base material, and a “base material (III)” was a glass base material. In FIG. 13, a “base material (IV)” to a “base material (VII)” were obtained by respectively sputtering titanium (Ti), zirconium (Zr), aluminum (Al), and platinum (Pt) on a circular cover glass under the same conditions as those of the samples S1 to S4 to have a three-dimensional nanostructure formed on the cover glass as a smooth substrate. A “base material (VIII)” to a “base material (XI)” respectively corresponded to the samples S1 to S4 described above or more specifically, were respectively obtained by transferring the NST-Ti, NST-Zr, NST-Al, NST-Pt described above onto a circular cover glass.

FIG. 13 shows the results of staining the cells seeded on each of the base materials (the “base material (I)” to the “base material (XI)”), with rBC2LCN-FITC (manufactured by FUJIFILM Wako Pure Chemical Corporation) on the seventh day after seeding. This reagent allows only the undifferentiated iPS cells to be stained. Placing a cell face downward on even an opaque base material allows for observation by bn inverted microscope. In the culture using the “base material (I)” that was the control under culture conditions (more specifically, under general undifferentiated culture conditions of iPS cells), the iPS cells were grown to be spread on a horizontal plane, and colonies of the undifferentiated cells were stained homogeneously in the entire field of view. In the culture using the “base material (II)” and the “base material (III)” that were the ECM-uncoated plates for tissue culture, on the other hand, substantially no cells were observed because the iPS cells did not survive but died due to anoikis or because living cells were removed by replacement of a culture medium. In the culture using the “base material (IV)” to the “base material (VII)” that respectively had films of three-dimensional nanostructures formed on the surface thereof by sputtering, survival or growth of cells was not at all observed under the ECM uncoated condition. In the culture using the “base material (VIII)” to the “base material (XI)” (the samples S1 to S4) that were the fiber structures having the three-dimensional nanostructures on the surface thereof, on the contrary, survival and growth of the iPS cells in a dome-like shape were observed even under the ECM uncoated condition.

According to the results described above, it is thought that the effect of allowing the iPS cells to survive and to be grown by using the “base material (VIII)” to the “base material (XI)” is not simply achieved by only the characteristics of the metal or the metal compound present on the surface of the base material and the three-dimensional nanostructure having the microscopic irregularities at the nano level formed by sputtering. More specifically, it is thought that satisfying such conditions as the fibrous form, the three-dimensional mesh structure configured by crossing such fibers or the like, as well as the three-dimensional nanostructure formed on the surface of the respective fibers and configured by the metal-containing particles including the metal or the metal compound described above provides a three-dimensional conformation and an electrostatic distribution as if simulating the ECM molecules having the RGD sequence and thereby provides the growth surface desired for the pluripotent stem cells.

### (Reseeding iPS Cells Grown on NST and Confirming iPS Cells Kept in Undifferentiated State)

The procedure performed culturing and reseeding of the iPS cells by using the “base material (VIII)” to the “base material (XI)” (the samples S1 to S4) and confirmed that the iPS cells were kept in the undifferentiated state. The iPS cells grown for one week (first passage) on one of the “base material (VIII)” to the “base material (XI)” (process D of FIG. 5) were dissociated from the base material by a method similar to the method employed at the time of the culture of the first passage, and were then reseeded on both a plate for tissue culture coated with iMatrix-511 (the same base material as “base material (I)”) and on the same base material as that for the first passage (one of the “base material (VIII)” to the “base material (XI)”) (process F and process E of FIG. 5). After the reseeded cells were cultured for one week with replacement of the culture medium every day, the procedure checked whether the cultured cells were kept in the undifferentiated state by using rBC2LCN-FITC.

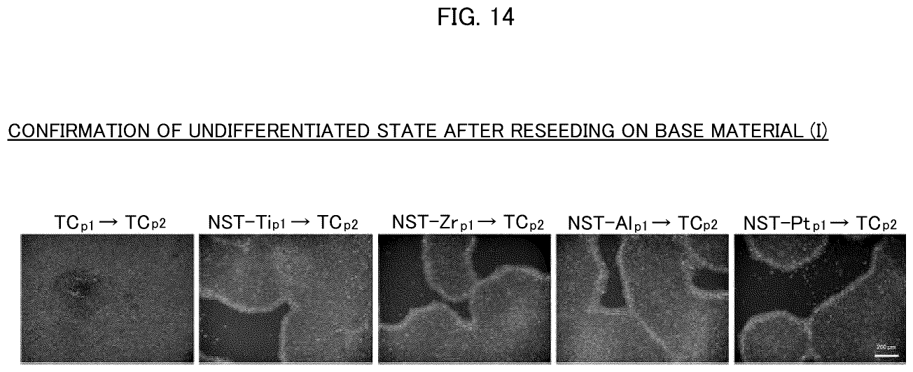

FIG. 14 is an explanatory diagram showing the results of checking whether the iPS cells were kept in the undifferentiated state after culturing for one week (first passage/p1) using one of the “base material (VIII)” to the “base material (XI)” (process D of FIG. 5), reseeding on the “base material (I)”, and further culturing for another one week (second passage/p2) (process F of FIG. 5). In FIG. 14, the result of culturing of the first passage (p1) using the base material (VIII) is shown as “NST-Tiₚ₁→TCₚ₂”; the result of culturing of the first passage (p1) using the base material (IX) is shown as “NST-Zrₚ₁→TCₚ₂”; the result of culturing of the first passage (p1) using the base material (X) is shown as “NST-Alₚ₁→TCₚ₂”; and the result of culturing of the first passage (p1) using the base material (XI) is shown as “NST-Ptₚ₁→TCₚ₂”. FIG. 14 also shows the result of using the “base material (I)” for both the first passage and the second passage as “TCₚ₁→TCₚ₂”. As shown in FIG. 14, in the case of reseeding on the ECM-coated “base material (I)”, it was confirmed that the iPS cells were grown with being kept in the undifferentiated state as normal iPS cells, regardless of whether the base material used for the first passage was the “base material (VIII)”, the “base material (IX)”, the base material (X)” or the “base material (XI)” and regardless of whether the metal of the metal-containing particles in the base material for the first passage was Ti, Zr, Al, or Pt.

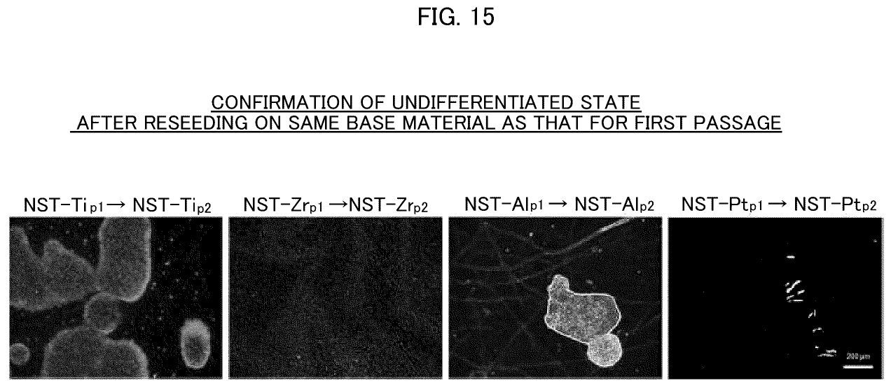

FIG. 15 is an explanatory diagram showing the results of checking whether the iPS cells were kept in the undifferentiated state after culturing for one week (first passage/p1) using one of the “base material (VIII)” to the “base material (XI)” (process D of FIG. 5), reseeding on the same base material as that used for the first passage (process E of FIG. 5) and further culturing for another one week (second passage/p2) (process F of FIG. 5). In FIG. 15, the result of using the base material (VIII) is shown as “NST-Tiₚ₁→NST-Tiₚ₂”; the result of using the base material (IX) is shown as “NST-Zrₚ₁→NST-Zrₚ₂”; the result of using the base material (X) is shown as “NST-Alₚ₁→NST-Alₚ₂”; and the result of using the base material (XI) is shown as “NST-Ptₚ₁→NST-Ptₚ₂”. As shown in FIG. 15, in the case of reseeding on the same base material as that for the first passage, the result on the “base material (VIII)” (NST-Ti) shows the favorable growth and the undifferentiated state of the cells. The result of the “base material (X)” (NST-Al) shows the growth of colonies of undifferentiated iPS cells after the culture of the second passage, although the growth volume is smaller than the result of the “base material (VIII)”. The results of the “base material (IX)” (NST-Zr) and the “base material (XI)” (NST-Pt) substantially shows no growth of the cells after the culture of the second passage. Based on these results, it is thought that NST-Ti and NST-Al are preferable as the base material used for the growth of the first passage and that NST-Ti is more preferable as the base material used for the continuous culture of two or more passages.

### (Confirming iPS Cells Kept in Undifferentiated State After Repeated Passage on NST-Ti)

The procedure performed passage culture for a longer time period using the base material (XIII) (NST-Ti), which is a base material used for culturing iPS cells without a protein-containing coating material and which is a base material thought to be suitable for the continuous culture of two or more passages, and checked the repeated passage and the undifferentiation. More specifically, after culturing on NST-Ti for one week (process D of FIG. 5), the procedure dispersed the iPS cells into single cells by the dispersion method described above, reseeded the single iPS cells on NST-Ti (process E of FIG. 5), and cultured the reseeded iPS cells for one week. After the culture for one week, the procedure confirmed that the iPS cells were sufficiently grown and were kept in the undifferentiated state by staining with rBC2LCN-FITC, and repeated an operation of dispersing the same sample again and reseeding the dispersed cells on NST Ti (process E of FIG. 5) up to the tenth passage. In the description below, culture of n-th passage on NST-Ti is expressed as “P₍ₙ₎”.

The procedure performed an operation of reseeding on NST Ti and an operation of seeding on an ECM-coated plate for tissue culture (base material (I)) for confirmation of the undifferentiated state (process F of FIG. 5) after confirmation of the undifferentiated state by staining with rBC2LCN-FITC in each of the passages (P₍ₙ₎) of culture on NST-Ti. After culture in a plate for checking the undifferentiated state (P₍ₙ₊₁₎), the procedure performed detailed morphologic observation by using a phase contrast microscopic image and an rBC2LCN-FITC-stained phase contrast microscopic image, so as to check whether the cultured iPS cells were kept in the state characteristic for undifferentiated iPS cells.

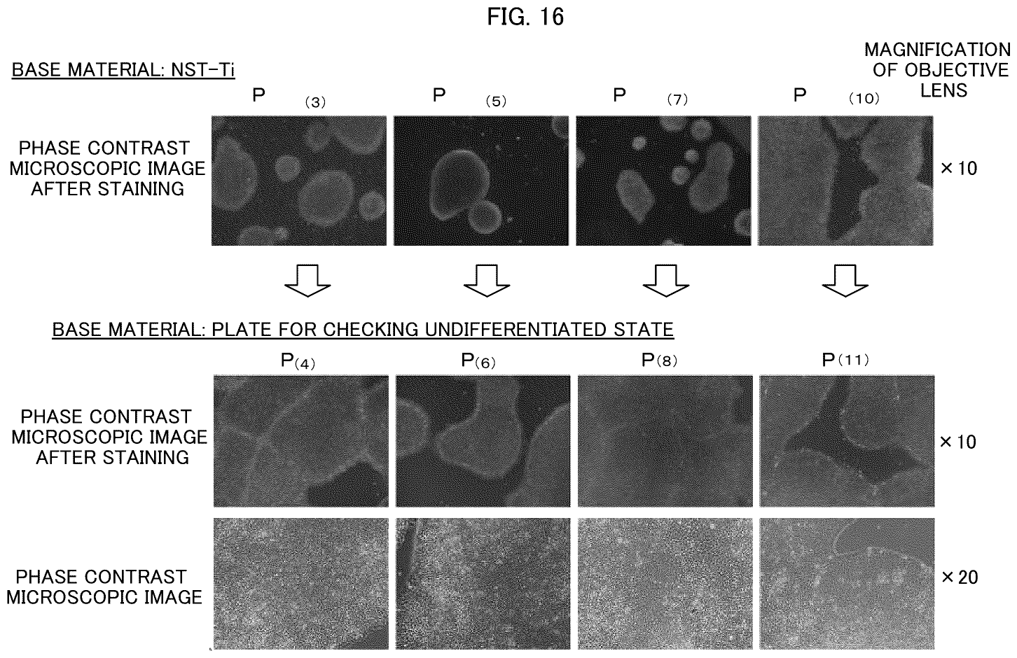

FIG. 16 is an explanatory diagram showing the results of checking the undifferentiated state in phase contrast microscopic images after staining with rBC2LCN-FITC in each of the passages (P₍ₙ₎), as well as the results of subsequently performing culturing on a plate for checking the undifferentiated state and examining the adhesion and growth of cells by phase contrast observation and the results of verifying the undifferentiation by staining with the rBC2LCN-FITC (shown as P₍ₙ₊₁₎) in FIG. 16). As shown in FIG. 16, it was confirmed that the iPS cells were grown on NST Ti with being kept in the undifferentiated state, in any passage number. The iPS cells grown on NST-Ti in each passage number were dissociated and seeded on a plate for checking the undifferentiated state and were then subjected to detailed microscopic observation. In a phase contrast microscopic image after staining with rBC2LCN-FITC, the expression of an undifferentiation marker on the cell surface was observed. In a high-magnification (20-fold object lens magnification) phase contrast microscopic image, the iPS cells morphologically kept in the undifferentiated state (more specifically, the iPS cells having large nuclei with scanty cytoplasm and nuclear bodies of clear contrast and forming a colony of compactly and closely packed cells with clear boundaries between adjacent cells) were observed. As described above, in the case of using NST Ti as the base material, it was confirmed that even continuous culture up to the tenth passage allowed for the growth of the cells kept in the undifferentiated state.

The present disclosure is not limited to the above embodiment described above but may be implemented in a variety of configurations without departing from the subject matter or the scope of the present disclosure. For example, the technical features of the embodiment corresponding to the technical features of the respective aspects described in Summary may be appropriately changed, altered, replaced or combined, in order to solve part or the entirety of the problems described above or in order to achieve part or the entirety of the advantageous effects described above. The technical features may be appropriately omitted unless the technical features are mentioned as essential in the description hereof.

The present disclosure may be implemented by any of the following aspects:

### Aspect 1

There is provided a scaffolding substrate for pluripotent stem cells, which is formed as a fiber structure where fibers of textile are arranged three-dimensionally, wherein the textile has, on a surface thereof, a three-dimensional nanostructure in which metal-containing particles that are made of at least one of a metal and a metal compound and that have a particle diameter of not less than 1 nm and not greater than 60 nm, are arranged three-dimensionally.

### Aspect 2

In the scaffolding substrate for pluripotent stem cells described in the above aspect 1, the three-dimensional nanostructure may have a primary structure configured by the metal-containing particles having the particle diameter of not less than 5 nm and not greater than 10 nm; and a secondary structure configured by a cluster that is an aggregate of the metal-containing particles and that has a diameter of not less than 30 nm and not greater than 100 nm.

### Aspect 3

In the scaffolding substrate for pluripotent stem cells described in either the above aspect 1 or the above aspect 2, the fibers of the textile may have a diameter in cross section of not less than 100 nm and not greater than 700 nm.

### Aspect 4

In the scaffolding substrate for pluripotent stem cells described in any one of the above aspects 1 to 3, the fiber structure may be formed by two or more layers of the textile that are laid one upon another.

### Aspect 5

In the scaffolding substrate for pluripotent stem cells described in any one of the above aspects 1 to 4, an interval between the fibers of the textile configuring the fiber structure may be not less than 0.5 μm and not greater than 10 μm.

### Aspect 6

In the scaffolding substrate for pluripotent stem cells described in any one of the above aspects 1 to 5, the fibers of the textile may be formed in a tubular form or in a semi-tubular form.

### Aspect 7

In the scaffolding substrate for pluripotent stem cells described in any one of the above aspects 1 to 6, a metal element configuring the metal-containing particles may include at least one species among titanium (Ti), zirconium (Zr), aluminum (Al) and platinum (Pt).

### Aspect 8

In the scaffolding substrate for pluripotent stem cells described in any one of the above aspects 1 to 7, the metal element configuring the metal-containing particles may include at least one species out of titanium (Ti) and aluminum (Al).

### Aspect 9

In the scaffolding substrate for pluripotent stem cells described in any one of the above aspects 1 to 8, the metal element configuring the metal-containing particles may include titanium (Ti).

### Aspect 10

There is provided a cell culture vessel, comprising the scaffolding substrate for pluripotent stem cells described in any one of the above aspects 1 to 9.

### Aspect 11

There is provided a method of culturing pluripotent stem cells, comprising: a culture process of culturing pluripotent stem cells by using the scaffolding substrate for pluripotent stem cells described in any one of the above aspects 1 to 9.

What is claimed is:

1. A scaffolding substrate for pluripotent stem cells comprising a fiber structure including textile fibers arranged three-dimensionally, wherein:
the textile fibers comprise, at least on a surface thereof, a three-dimensional nanostructure in which metal-containing particles are arranged three-dimensionally, and
the metal-containing particles comprise at least one of a metal and a metal compound, and comprise a particle diameter of not less than 1 nm and not greater than 60 nm.

2. The scaffolding substrate for pluripotent stem cells according to claim 1, wherein:
the particle diameter of the metal-containing particles is not less than 5 nm and not greater than 10 nm; and
the three-dimensional nanostructure comprises:
a primary structure configured by the metal-containing particles; and
a secondary structure configured by a cluster of the metal-containing particles that are aggregated together, the cluster comprising a diameter of not less than 30 nm and not greater than 100 nm.

3. The scaffolding substrate for pluripotent stem cells according to claim 1,
wherein the textile fibers comprise a diameter in cross section of not less than 100 nm and not greater than 700 nm.

4. The scaffolding substrate for pluripotent stem cells according to claim 2,
wherein the textile fibers comprise a diameter in cross section of not less than 100 nm and not greater than 700 nm.

5. The scaffolding substrate for pluripotent stem cells according to claim 1,
wherein the fiber structure comprises two or more layers of the textile fibers that are laid one upon another.

6. The scaffolding substrate for pluripotent stem cells according to claim 2,
wherein the fiber structure comprises two or more layers of the textile fibers that are laid one upon another.

7. The scaffolding substrate for pluripotent stem cells according to claim 5,
wherein an interval between the textile fibers configuring the fiber structure is not less than 0.5 μm and not greater than 10 μm.

8. The scaffolding substrate for pluripotent stem cells according to claim 6,
wherein an interval between the textile fibers configuring the fiber structure is not less than 0.5 μm and not greater than 10 μm.

9. The scaffolding substrate for pluripotent stem cells according to claim 1,
wherein the textile fibers are formed in a tubular form or in a semi-tubular form.

10. The scaffolding substrate for pluripotent stem cells according to claim 2,
wherein the textile fibers are formed in a tubular form or in a semi-tubular form.

11. The scaffolding substrate for pluripotent stem cells according to claim 1,
wherein the metal-containing particles include at least one metal element selected from the group consisting of titanium (Ti), zirconium (Zr), aluminum (Al) and platinum (Pt).

12. The scaffolding substrate for pluripotent stem cells according to claim 2,
wherein the metal-containing particles include at least one metal element selected from the group consisting of titanium (Ti), zirconium (Zr), aluminum (Al) and platinum (Pt).

13. The scaffolding substrate for pluripotent stem cells according to claim 11,
wherein the metal-containing particles include at least one of titanium (Ti) and aluminum (Al).

14. The scaffolding substrate for pluripotent stem cells according to claim 12,
wherein the metal-containing particles include at least one of titanium (Ti) and aluminum (Al).

15. The scaffolding substrate for pluripotent stem cells according to claim 13,
wherein the metal-containing particles include titanium (Ti).

16. The scaffolding substrate for pluripotent stem cells according to claim 14,
wherein the metal-containing particles include titanium (Ti).

17. A cell culture vessel, comprising the scaffolding substrate for pluripotent stem cells according to claim 1.

18. A cell culture vessel, comprising the scaffolding substrate for pluripotent stem cells according to claim 2.

19. A method of culturing pluripotent stem cells, comprising:
culturing pluripotent stem cells provided on the scaffolding substrate for pluripotent stem cells according to claim 1.

20. A method of culturing pluripotent stem cells, comprising:
culturing pluripotent stem cells provided on the scaffolding substrate for pluripotent stem cells according to claim 2.
